(19) United States
(12) Patent Application Publication
(10) Pub. No.: US 2023/0372931 A1
(43) Pub. Date: Nov. 23, 2023

(54) SYSTEM AND METHOD FOR LOADING A MICROFLUIDIC CHIP

(71) Applicant: LUMICKS CA HOLDING B.V., Amsterdam (NL)

(72) Inventor: Myles McGarrigle, Amsterdam (NL)

(21) Appl. No.: 18/030,170

(22) PCT Filed: Oct. 7, 2021
(86) PCT No.: PCT/NL2021/050609

§ 371 Date: Apr. 4, 2023

(30) Foreign Application Priority Data
Oct. 7, 2020 (NL) 2026640

(51) Int. Cl. B01L 3/00

(52) U.S. Cl. CPC B01L 3/502723; B01L 2200/0684; B01L 2300/028; B01L 2400/0436; B01L 2400/0478

(57) ABSTRACT

A method of reducing or preventing bubbles in a microfluidic sample liquid is provided. The method comprises providing a microfluidic sample holder comprising an enclosed fluid channel for holding at least part of the sample liquid, and filling at least part of the fluid channel with a sample liquid. The method further comprises: a step of pressurizing the sample liquid in the fluid channel to raise a sample liquid pressure to an elevated pressure higher than an ambient pressure and an operating pressure, a step of maintaining the sample liquid pressure at least at the elevated pressure for a predetermined period to cause dissolving of gas into the sample liquid, and a step of reducing the sample liquid pressure from the elevated pressure to the operating pressure. An associated system is also provided.

### CROSS-REFERENCE TO RELATED APPLICATION(S)

This Application is a Section 371 National Stage Application of International Application No. PCT/NL2021/050609, filed Oct. 7, 2021, and published as WO 2022/075848 A1 on Apr. 14, 2022, and further claims priority to Netherlands Patent Application No. 2026640, filed Oct. 7, 2020.

### TECHNICAL FIELD

The present disclosure relates to systems and methods for loading a microfluidic chip, in particular for loading the chip with a fluid.

### BACKGROUND

Microfluidic experiments and the associated devices are well known, e.g. as so-called “lab-on-a-chip”.

A microfluidic device may comprise a sample holder or “chip” comprising a microfluidic channel and/or holding space for holding a sample in a fluid medium. The sample fluid may comprise plural components and/or one or more particles. Various properties of one or more portions of such sample may be studied using different means. Often, the studies require optical means such as one or more of microscopy and imaging which may comprise static and/or dynamic analysis such as image and/or video-analysis.

Video tracking and acoustic manipulation of biological cells has, e.g. been described in WO 2018/083193.

It has been found that optical detection methods may be complicated or rendered impossible when the sample fluid comprises gas bubbles which interfere with imaging. Gas bubbles may also reduce or destroy acoustic performance of the acoustic manipulation devices. In addition, bubbles are found to cause problems in cell culturing. In microfluidics, moreover, even small bubbles may form significant obstructions to flow and/or diffusion processes in the narrow channels involved.

Some properties to be studied may be one or more of time dependent, (critically) dependent on sample composition and/or (critically) dependent on particular fluid dynamics in the sample holder. Also such properties may be affected by gas bubbles in the sample in unwanted manner.

It has further been found that undesired gas bubbles may form in the sample after loading, in particular when the sample has been allowed to stay for some time for one or more of storage, incubation and development of one or more processes not expected to produce gas bubbles.

E.g., for some applications it may be required to flush live cells into the holding space and to allow them to bind to a wall surface of the holding space, e.g. forming a cell monolayer, by incubating or culturing the cells on the wall surface for a predetermined amount of time, e.g. a period of hours. If, in such process bubbles form or grow (e.g. associated with injection of the sample and/or by natural degassing during the culturing phase) these bubbles may interfere with the cell culturing and/or the formation of a monolayer. Such natural degassing may occur in particular if fluids are saturated with a gas. In particular, during culturing phase large bubbles may form due to biological processes and/or fusion of microbubbles (natural degassing) and these bubbles may be flushed through at least part of the holding space when medium (sample liquid) and/or sample is exchanged. This may cause damage to the cell monolayer.

Bubble prevention systems exist, e.g. using bubble traps based on recesses and/or semipermeable membranes. However, these may cause problems with one or more of clogging, pollution and infection, in particular when at least part of the sample comprises one or more biological components such as biological cells. Also, membranes may be non-compatible with cleaning procedures and reagents and/or may be fragile and prone to damage. Such systems prove also unsuccessful in relation to late-formed bubbles.

US 2017/0121663 discloses systems and methods for improved flow properties in fluidic and microfluidic systems. The system includes a microfluidic device having a first microchannel, a fluid reservoir having a working fluid and a pressurized gas, a pump in communication with the fluid reservoir to maintain a desired pressure of the pressurized gas, and a fluid-resistance element located within a fluid path between the fluid reservoir and the first microchannel. The fluid-resistance element includes a first fluidic resistance that is substantially larger than a second fluidic resistance associated with the first microchannel. US 2017/0121663 teaches that damage caused by bubbles is prevented, inhibited, or limited through use of automated fluid-flushing of the microfluidic devices using periodically increased flow rates.

Yet another approach is chemical treatment of the sample holder, e.g. relying on aggressive cleaning reagents such as acids and bleach. Such substances may be incompatible with particular sample holder materials and/or with delicate sample components such as biological cellular and/or subcellular structures.

Improved methods and devices for reduction or prevention of bubbles and/or bubble formation are therefore desired.

### SUMMARY

In view of the above, a method of reducing or preventing bubbles in a microfluidic sample liquid is provided. The method comprises providing a microfluidic sample holder comprising an enclosed fluid channel for holding at least part of the sample liquid, and filling at least part of the fluid channel with a sample liquid.

The method further comprises:
a step of pressurizing the sample liquid in the fluid channel to raise a sample liquid pressure to an elevated pressure higher than an ambient pressure and an operating pressure,
a step of maintaining the sample liquid pressure at least at the elevated pressure for a predetermined period to cause dissolving of gas into the sample liquid, and
a step of reducing the sample liquid pressure from the elevated pressure to the operating pressure.

The entire fluid channel may be filled with the sample liquid.

It is noted that pressurizing a liquid containing bubbles may inherently reduce bubble size by compressing their volume and increasing the gas pressure inside the bubbles. However, in such case the amount of gas contained in a bubble, e.g. number of particles (e.g. atoms and/or molecules) in the gas phase, remains substantially equal and releasing the pressure to an initial pressure returns the bubbles to their initial size.

However, it has been found that by pressurizing the sample liquid to an elevated pressure (herein also referred to as “overpressure”) and maintaining the sample liquid for prolonged duration at at least the elevated pressure, gas from the gas bubbles can be made to dissolve into the liquid so that gas bubbles are made to effectively disappear.

Without wishing to be bound by any particular theory, it is considered that the gas or gases may be made to dissolve substantially fully into the liquid and bubbles, including microbubbles containing only very few atoms or molecules, disappear. The dissolution may contain that particles (e.g. atoms and/or molecules) associated with the bubbles become accommodated individually and separately in the liquid phase so that bubbles reduce in size by reduction of the number of particles in the gas phase. A bubble may be considered to be fully disappeared when no boundary between a liquid phase and a gas phase may be recognised. By decreasing size and/or disappearance of bubbles nucleation sites for (further) bubble formation and/or bubble growth are considered to be reduced and/or destroyed. Also, all surfaces in contact with the liquid such as below a liquid level may become fully wetted.

It has been found that bubbles may not reappear after reducing the pressure removing at least part of the overpressure. In particular, bubbles may remain absent or at least undetectable for prolonged periods, longer than time scales of interest in experiments and/or manipulation of the sample liquid or samples comprising at least part of the sample liquid.

It is noted that the present method contrasts “forced degassing” wherein pressure is temporarily reduced (or temperature is temporarily raised) and bubble formation is increased, at least for some time, to remove (potential) gas particles (e.g. atoms and/or molecules) from the liquid into the bubbles, which may thereafter be removed by displacement or rupture. By forced degassing nucleation sites are exploited and/or promoted, in contrast to the present concepts wherein nucleation sites are suppressed and/or destroyed. Forced degassing fluids prior to use in microfluidic devices may work for bubble prevention, but it may not be compatible with experiments and/or methods depending on fluids with dissolved gasses (such as for example live biological cell experiments). Cells may depend on dissolved gasses, e.g. oxygen, for their survival and removal of those gasses from the medium may therefore be incompatible with biological cell experiments.

The presently provided concepts retain such gases.

The operating pressure may be equal to the ambient pressure. This facilitates operation.

Filling the channel may be done at a filling pressure, which may be different from the operating pressure and/or ambient pressure. Then, the elevated pressure may preferably be also higher than the filling pressure.

The filling pressure and/or the operating pressure may be ambient pressure and/or a pressure that is at or near a pressure for a particular processing step in an experiment using the sample. E.g. the operating pressure may be determined as a sample liquid pressure at which the sample is used for study, measurement, manipulation and/or other use. E.g., in a sample comprising cellular bodies, the cellular bodies may execute and/or be made to execute particular biological functions as in an environment that would be natural for them. Also or alternatively, the filling pressure and/or the operating pressure may be slightly higher or lower than ambient pressure to force the sample liquid into and through the at least part of the channel, e.g. by a pressure difference with respect to ambient pressure such as by pushing and/or by suction.

Between the step of filling at least part of the fluid channel and the step of pressurizing the sample liquid, the method may comprise an intermediate step of storing the sample holder containing the sample liquid for a predetermined period at the ambient pressure.

Such intermediate step may comprise accommodating and/or causing a development of or in the sample liquid and/or any component thereof, such as one or more of reaction, separation, phase change, incubation, developing, settling, equilibration, etc.

At least one embodiment may comprise providing an amount of gas in a limited volume in contact with the sample liquid providing a liquid level. Then at least one of the steps of pressurizing the sample liquid in the fluid channel and maintaining the sample liquid pressure at or above the elevated pressure for a predetermined period may comprise compressing the gas in the volume, in particular comprising compressing the gas to pressurize the liquid.

Thus, a larger static and/or dynamic range for establishing and/or detection of the sample liquid pressure may be achieved. The amount of gas may have one or more of a predetermined volume, predetermined pressure and/or predetermined composition which may be associated with the sample holder and/or the sample liquid. A gas pressure may be measurable more easily and reliably than a liquid pressure. A liquid level may be readily detectable which may facilitate pressure detection due to shift in position of the liquid level with respect to a container and/or the channel.

Compressing the gas to pressurize the liquid provides for indirect compression, such as preventing compression by deforming a container portion. This may e.g. reduce a position shift of at least part of the liquid, contamination and/or damage of the liquid and/or of the sample holder and/or it may facilitate construction of the sample holder.

Also or alternatively, compressing a gas may be done easily and reliably and/or in a more controlled way compared to pressurising a liquid directly.

At least one embodiment of the method may comprise adding an additional amount of sample liquid to the known amount of sample liquid, preferably by addition into a reservoir in fluid connection with the fluid channel.

It has been found that once a first liquid is (nearly) bubble free (which may include substantially full wetting of surfaces below a liquid level) and further liquid is added to the first liquid, that the further liquid and/or the thus resulting liquid combination or liquid mixture may also remain bubble free, at least easier and/or for longer than when the first liquid were not prior subject to at least the steps of pressurizing the sample liquid in the fluid channel and maintaining the sample liquid pressure at or above the elevated pressure for the predetermined period. Note that a liquid combination or mixture may be a homogeneous mixture or at least partly a two-phase mixture wherein regions of one liquid and of another liquid may be separated and individually identifiable.

The elevated pressure may be in a range of about 1-20 Bar (100-2000 kPa) over ambient pressure, in particular in a range of about 2-10 Bar (200-1000 kPa), more in particular in a range of about 3-8 Bar (300-800 kPa) such as between about 4 and 7 Bar (400-700 kPa).

However, the step of maintaining the sample liquid pressure at or above the elevated pressure for a predetermined period to cause dissolving of gas into the sample liquid may comprise maintaining the sample liquid pressure at or above the elevated pressure until bubbles are reduced and/or have disappeared to below a predetermined level, in particular below a predetermined level of detection. Also or alternatively, the step may comprise maintaining the sample liquid pressure at or above the elevated pressure for a period of at least 15 minutes, preferably at least 30 minutes such as 40-60 minutes; and preferably less than 2 hours, more preferably less than 1.5 hours such as less than 1 hour. The duration may be determined from (statistical analysis of) previous practice and/or experiment, e.g. determined on the basis that bubbles do not remain and/or reappear for more than 90% or 95% or >3 standard deviations from average of the sample holders on which the method is used.

Suitable periods may be associated with accommodating and/or causing a development of or in the sample liquid and/or any component thereof, such as one or more of reaction, separation, phase change, incubation, developing, settling, etc. Also or alternatively suitable periods may be associated with personnel activities such as pauses and or working hours such as maintaining the sample liquid pressure at or above the elevated pressure overnight and/or during a weekend. The time required to dissolve trapped gas or microbubbles (thereby reduce the available nucleation points for later bubble formation) may be further reduced by using degassed fluids in this step. After the removal of existing bubbles or gas pockets by pressurization the fluid may be mixed with and/or exchanged for non-degassed fluids (e.g. cell culture medium) if required. Note that such exchange may be done by continuous replacement (flushing) of one liquid by another for reducing risks of reintroducing and/or creation of bubbles. Bubble formation and/or growth are suppressed also in the mixed and/or replaced liquids.

A predetermined level of detection, if used, may be determined by detection and/or measurement apparatus (to be) used for experiments and/or measurements and/or operation of the sample holder and at least part of the sample liquid therein. The detection level may be determined as “undetectable”, or at least undetectable within acceptable noise and/or tolerances at operation settings used for average and/or intended experiments and/or measurements and/or operation of the sample holder and at least part of the sample liquid therein. E.g., sufficient duration may be determined by detecting and identifying a bubble signal associated with a bubble in the sample liquid and determining reduction and/or disappearance of the identified bubble signal to below a detection limit. In a particular example a bubble signal may be optical detectability of the nature “a bubble is (still) or is not (anymore) visible”, in particular optical imaging conditions such as at a particular magnification and/or illumination. Also or alternatively an acoustic signal may be used such as detecting (variations in) a sound velocity profile and/or (variations in) a particular resonance frequency property of an acoustic wave generated in the sample holder containing the sample liquid. The skilled reader will be able to determine other detection methods such as detection and/or determination of homogeneity of and/or interruption in the sample liquid.

The sample liquid may comprise sample particles, in particular biological cellular bodies.

Also or alternatively, at least part of the channel may be provided with a functionalized wall surface portion. A functionalized wall surface portion, in particular in combination with a sample comprising biological cellular bodies, allows studying and/or exploiting particular interactions between the substance(s) providing the functionalization and sample components. Various examples of suitable functionalized wall surface portions, cellular bodies and associated studies are disclosed in WO 2018/083193, incorporated herein by reference.

Part of the channel may be identified as a holding space. At least part of the functionalized wall surface portion may be formed in the holding space. The holding space may be determined by a structural and/or geometric boundary and/or transition in the sample holder such as a change in size and/or shape of at least part of the channel. Also or alternatively the holding space may be determined by one or more of manipulation and/or detection apparatus associated with the sample holder, such as a window and/or another optical element and/or detail, a signal generator, a connector etc.

At least one method may comprise providing a signal generator for generating an acoustic wave in the sample holder, and providing, using the signal generator, a driving signal to the sample holder generating an acoustic wave in the sample holder, in particular being configured for providing an acoustic force in at least part of the channel, in particular providing an acoustic force for manipulating one or more objects in the sample liquid.

Thus, at least part of the sample liquid and a possible particle therein may be subjected to the acoustic wave for study and/or manipulation, without being affected by bubbles. Also or alternatively, the acoustic wave could be used for detection of a bubble in the liquid. Moreover, since a bubble is likely to have a different compressibility than liquid surrounding it, the acoustic wave, in particular an acoustic force, may be used for detection and/or manipulation of a bubble itself.

Also or alternatively, at least one method may comprise providing an optical trapping beam (commonly referred to as optical tweezers) for trapping and/or manipulating at least part of a sample in at least part of the channel.

Optical tweezers and associated techniques have proven to be very versatile for study and/or manipulation of particles in a sample comprising a sample liquid, and bubbles may negatively affect quality of the optical trapping beams, e.g. causing one or more of absorption, dispersion, scattering and deflection. Also, bubbles in the sample fluid may cause oscillations and/or unwanted motion of the sample fluid which may cause spurious forces on objects in the optical traps thereby reducing the amount of control over the applied and/or measured force. The present method therefore assists increasing reliability of optical tweezers experiments and/or -use.

Further, associated with the foregoing, a system for filling a microfluidic sample holder is provided. In the system, the sample holder comprises an enclosed microfluidic channel provided with a liquid inlet and a liquid outlet and a filling system for filling at least part of the microfluidic channel with a sample liquid. The filling system is configured for controllably pressurizing a sample liquid in the fluid channel to raise a sample liquid pressure to an elevated pressure higher than an ambient pressure and an operating pressure, and maintaining the sample liquid pressure at least at the elevated pressure for a predetermined period to cause dissolving of gas into the sample liquid for removing and/or preventing gas bubbles from the sample liquid, and controllably reducing the sample liquid pressure from the elevated pressure to the operating pressure.

The system thus allows filling the sample holder with a sample liquid and removing bubbles from the liquid, for experimenting and/or use etc. of the sample, unhindered by bubbles.

The sample holder may comprise a holding space for holding at least part of the sample.

The system may comprise a sealed or sealable gas reservoir for providing a defined amount of gas in a limited volume in contact with the sample liquid providing a liquid level.

The gas reservoir may have a predetermined size and/or comprise at least one structure for defining the liquid level at a predetermined position. The better the amount of gas is known the better the sample liquid pressure may be known and/or be determinable. The amount of gas may be determined by the volume of the gas reservoir. The gas reservoir may be openable to allow and/or release gas and/or to define a gas pressure.

The filling system may comprise a compressor configured to compress a gas in the gas reservoir to pressurize the liquid, e.g. comprising a piston in contact with the gas.

As indicated above, compressing a gas may facilitate application of a pressure to the sample liquid. Also, construction of the system may be facilitated. A piston facilitates a pressure increase by reducing volume. Note that a piston area (e.g. diameter of a round piston) may be scaled in relation to an area of the liquid level (e.g. diameter of the liquid channel) so as to adapt adjustability of the pressurising and/or sensitivity of a pressure detection, etc.

The sample holder may be arranged, in use, with the channel substantially horizontal. The system may comprise a liquid reservoir in fluid communication with the channel for filling the channel and/or, in use, establishing a liquid level above the channel; e.g. the reservoir extends at least in part at a non-zero angle to the channel. The reservoir may have one or more of a size, shape, structure and construction material significantly different from the channel, e.g. being a module (to be) reversibly connected with the channel, e.g. at an inlet and/or out outlet of the channel. The reservoir may serve for a buffer volume of the sample liquid. This construction may facilitate manual filling and/or exchange of fluids e.g. using standard pipettes. Typical microfluidic channels may have a cross sectional area of below about 5 mm² and/or having a largest open length in cross section to a flowing direction (e.g. a diameter) of about 3 mm or below. The holding space may have a capacity on the order of tens of microliters or below. The reservoir may have a volume on the order of milliliters.

E.g., the system may comprise a liquid reservoir in fluid connection with the channel, wherein the reservoir defines a filling direction and at least part of the liquid reservoir comprises a section having an inclined wall section facing towards the filling direction, in particular a tapering section, e.g. a conical or flaring reservoir widening upward.

The inclined wall and/or tapering section may preferably be configured to, in use, be directed facing upward. An inclined wall may assist removal of bubbles since bubbles which tend to form at and/or stick to surfaces to escape into the liquid and towards a liquid surface without being hindered and/or recaptured by the wall surface.

The inclined wall and/or tapering section may define a section having a first inclination and/or first tapering angle, respectively, and a second, different inclination and/or second, different tapering angle, respectively. This may facilitate filling the sample holder to a particular level and/or promoting bubble formation in one or more dedicated locations in the sample holder.

Such liquid reservoir may also be suitably employed on its own, i.e. without requiring (method steps of) pressurisation of the sample liquid.

The sample holder, in particular a liquid reservoir thereof which is in fluid connection with the channel, may comprise a translucent or transparent portion for optical detection, in particular visual detection, of a liquid level in the reservoir.

Such sample holder facilitates performing the method disclosed herein. The sample holder may otherwise be at least partially opaque. A level mark may be provided for reference and reliability e.g. to facilitate quantitative control over liquid levels used in operation.

A light guide may operate on the basis of total internal reflection and/or on the basis of reflecting portions reflecting light back into and further along the light guide. It may also be based on gradient index principles such as known for gradient index lenses and gradient index fibers. Using a light guide increases freedom of design.

The translucent or transparent portion may comprise and/or be provided with one or more optical elements such as lenses, mirrors, filters etc. which may help increase detectability of the liquid level. For example, a lens may be integrated into a translucent portion made from a single material simply by providing a suitable curvature on the inside (fluid side) or outside (air side) of the translucent portion, i.e. by designing its geometry instead of integrating discrete optical elements of different materials.

The sample holder may comprise an at least partly opaque portion at or near the liquid inlet and/or outlet, wherein the opaque portion provides a translucent, possibly transparent, portion and/or an aperture, for lighting at least one of a liquid inlet of the channel, a liquid outlet of the channel, a reservoir (if present) and a level mark (if present). At least part of such opaque portion may be formed by a housing.

In particular in case of a sample holder having an inclined wall and/or tapering section as indicated above, the translucent, possibly transparent, portion and/or an aperture may be separate from, and possibly substantially opposite to, the translucent or transparent portion for optical detection.

In such sample holder at least part of the sample holder may be constructed to facilitate optical accessibility and/or optical inspection (wherein optical possibly includes visual).

Such sample holder may also be suitably employed on its own, i.e. without requiring (method steps of) pressurisation of the sample liquid.

Note that herein, “visual” means optical by the naked eye or minimally assisted eye (such as drawing assistance from one or more of contact lenses, spectacles, single lenses or loupes, hand-held infrared-viewer and handheld UV-light viewer) but without requiring stationary optical devices like microscopes and/or contrast imagers. In particular visual inspection may rely on providing a direct line of sight to the target and/or detail to be inspected.

A gas reservoir and a liquid reservoir may be combined in one reservoir. The system may comprise plural gas reservoirs and/or plural liquid reservoirs and/or plural combined gas and liquid reservoirs.

The system may comprise a closure for closing the gas reservoir and/or the liquid reservoir at least liquid tight and in particular gas tight, preferably releasably closing.

By gas tight closing a liquid reservoir it may be turned into a gas reservoir and/or a combined gas and liquid reservoir. A screwed closure (threaded and/or bayonet-locked) may be suitable and may resist a significant overpressure. A stop (“cork” or some other plug or cap) may be sufficient for low overpressures but may leak or even be “blown off” at higher overpressures. A releasable closure, e.g. a screw lid, may facilitate closing and replenishing.

The sample holder may comprise an acoustic wave generator for generating an acoustic wave in at least part of the channel. Also or alternatively, the system may comprise at least one optical tweezer for trapping and/or manipulating at least part of a sample in at least part of the channel.

Such system facilitates and/or improves one or more of detection, study, manipulation and use of at least part of a sample in the channel.

### BRIEF DESCRIPTION OF THE DRAWINGS

The above-described aspects will hereafter be more explained with further details and benefits with reference to the drawings showing a number of embodiments by way of example.

FIG. 1 is a schematic drawing of an embodiment of a manipulation system;

FIG. 2 is a schematic drawing of a sample holder for the system of FIG. 1;

FIG. 2A is a schematic detail of FIG. 2 as indicated;

FIGS. 3-11 show an exemplary workflow according to the presently provided concepts;

FIG. 12 is a schematic overview of method steps according to an embodiment;

FIG. 13 shows an exemplary sample holder assembly for the system and method in perspective;

FIG. 14 is a side view of the sample holder 300;

FIG. 15 is a bottom view of the sample holder 300;

FIG. 16 is a perspective cross section view of the sample holder assembly of FIGS. 13-15;

FIG. 17 shows part of the sample holder assembly of FIGS. 13-15;

FIG. 18 is a view like FIG. 17, but partly cut away;

FIG. 19 indicates a schematic cross section profile of the reservoir.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic suffixes.

Further, unless otherwise specified, terms like “detachable” and “removably connected” are intended to mean that respective parts may be disconnected essentially without damage or destruction of either part, e.g. excluding structures in which the parts are integral (e.g. welded or moulded as one piece), but including structures in which parts are attached by or as mated connectors, fasteners, releasable self-fastening features, etc. The verb “to facilitate” is intended to mean “to make easier and/or less complicated”, rather than “to enable”.

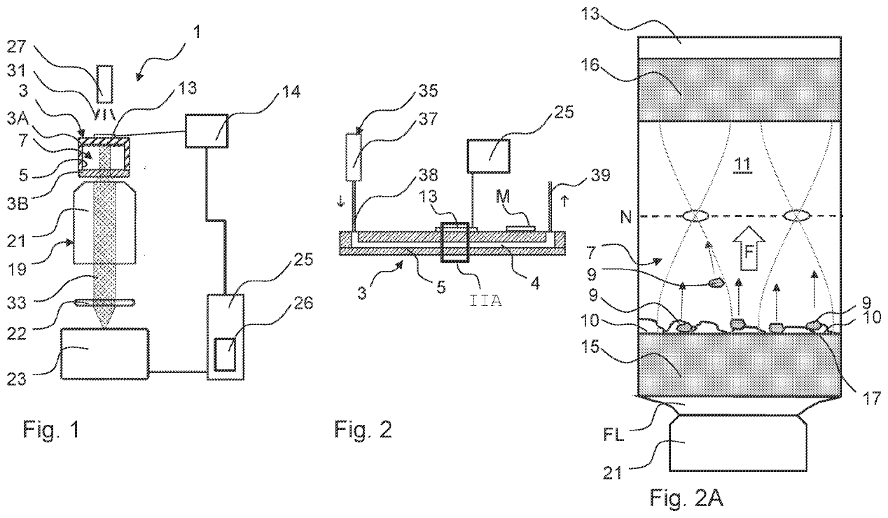

FIG. 1 is a schematic drawing of a manipulation system 1, FIG. 2 is a cross section of a sample holder and FIG. 2A is a detail of the sample holder of FIG. 2 as indicated with “IIA”.

The system 1 comprises a microfluidic sample holder 3 comprising a fluid channel 4 providing a holding space 5 for holding a microfluidic sample 7. As shown in FIG. 2A and further, the sample may typically comprise one or more objects like biological cellular bodies 9, 10 in a sample liquid 11 as exemplary objects of interest. It is noted that also, or alternatively, other types of objects like microspheres could be used, possibly attached to biological cellular bodies 9. The system 1 further comprises an acoustic wave generator 13, e.g. a piezo element, connected with the sample holder 3 to generate an acoustic wave in the holding space 5 exerting a force on the sample 7 and cellular bodies 9, 10 in the sample 7. The acoustic wave generator 13 is connected with an optional controller 14 and power supply, here as an option being integrated.

The sample holder 3 comprises a wall 15 providing the holding space 5 with an optional functionalized wall surface portion 17 to be contacted, in use, by part of the sample 7. Here, the functionalized wall surface portion 17 is provided with the cellular bodies 10 adhered to the surface of the wall 15, possibly with one or more primer layers in between (not shown). As explained in detail below, interaction of the cellular bodies 9 (and/or other objects) with the cellular bodies 10 may be studied with such system. A further wall, e.g. opposite wall 16, may also or alternatively be provided with a (further) functionalized wall surface portion.

The shown manipulation system 1 comprises a microscope 19 with an optional optical system such as an (optionally adjustable) objective 21 and a camera 23 connected with a computer 25 comprising a controller and a memory 26; more or less optical detectors and/or detectors of other types may be provided. The computer 25 may also be programmed for tracking one or more of the cellular bodies based on signals from the camera 23 and/or for performing microscopy calculations and/or for performing analysis associated with (super resolution) microscopy and/or video tracking, which may be sub-pixel video tracking. The computer or another controller (not shown) may be connected with other parts of the system 1 (not shown) for controlling at least part of the microscope 19 and/or another detector (not shown). In particular, the computer 25 may be connected with one or more of the acoustic wave generator 13, the power supply thereof and the controller 14 thereof, as shown in FIG. 1. The computer may also be connected to one or more fluidics valves, pressure- and/or flow sensors, pressure- and/or flow regulators, etc. in order to facilitate control over sample liquid flows.

The system further comprises an optional light source 27. The light source 27 may illuminate the sample 7 using any suitable optics (not shown) to provide a desired illumination intensity and intensity pattern, e.g. plane wave illumination, Köhler illumination, etc., known per se. Here, in the system light 31 emitted from the light source 27 is directed through the acoustic wave generator 13 to (the sample 7 in) the sample holder 3 and sample light 33 from the sample 7 is transmitted through the objective 21 and through an optional ocular 22 and/or other optional optics (not shown) to the camera 23.

The sample light 33 may comprise light 31 affected by the sample (e.g. scattered and/or absorbed) and/or light emitted by one or more portions of the sample 7 itself e.g. by fluorophores attached to the cellular bodies 9 or e.g. generated by bio-, or chemo-luminescence.

As shown in FIGS. 1 and 2, the sample holder 3 may comprise a part 3A that has a recess being, at least locally, generally U-shaped in cross section and a cover part 3B to cover and close (the recess in) the U shaped part providing a channel 4 and a holding space 5 enclosed in cross section.

The sample holder 3 is a microfluidic device of the type commonly referred to as a lab-on-a-chip. The sample holder may be a substantially planar device. At least part of the sample holder may be formed by a single piece of material with the channel 4 inside, e.g. glass, injection moulded polymer, etc. (not shown) or by fixing different layers of suitable materials together more or less permanently, e.g. by welding, glass bonding/direct bonding, gluing, taping, clamping, etc., such that the channel 4 and holding space 5 are formed in which the sample 7 may be contained, at least during the duration of an experiment. Forming the sample holder from a single piece of material may have the advantage that it forms an efficient acoustic cavity which enables the generation of high acoustic forces at the functionalized wall. Thus, a monolithic sample holder, at least at the location of the acoustic wave generator 13, may be preferred over an assembled sample holder for improving acoustic coupling, reducing losses and/or preventing local variations.

Embodiments of a sample holder 3 will be detailed below.

As shown in FIG. 2, the sample holder 3 is connectable to a fluid flow system 35 for introducing fluid into the holding space 5 of the sample holder 3 and/or removing liquid from the holding space 5, e.g. for flowing liquid through the channel 4 and holding space 5 (see arrows in FIG. 2).

The fluid flow system 35 may comprise a manipulation and/or control system, possibly associated with the computer 25. The fluid flow system 35 may comprise one or more of reservoirs 37, pumps, valves, and inlet conduits 38 for introducing one or more liquids and outlet conduits 39 for removing one or more liquids, sequentially and/or simultaneously. The sample holder 3 and the fluid flow system 35 may comprise connectors, which may be arranged on any suitable location on the sample holder 3, for coupling/decoupling without damaging at least one of the parts 3, 35, and preferably for repeated coupling/decoupling such that one or both parts 3, 35 may be reusable thereafter. Further, an optional machine-readable mark M or other identifier is attached to the sample holder 3, possibly comprising a memory.

FIG. 2A is a schematic of cellular bodies 9 in the sample holder 3 of FIG. 2. Part of the wall 15 of the sample holder 3 is optionally provided with a functionalized wall portion 17, e.g. an area of the area being covered with biological cells 10 of a different type to which the cellular bodies of interest 9 may adhere. Also shown is part of the microscope lens 21 and an optional immersion fluid layer FL for improving image quality.

On providing a periodic driving signal to the acoustic wave generator 13 a standing wave is generated in the sample holder 3. The standing wave exerts an acoustic force on objects 9, 10 in the sample liquid 11 having a different compressibility (also referred to as acoustic index) than the surrounding sample liquid 11. The signal is selected such that an antinode of the wave is generated at or close to the wall surface (of the sample holder 3 e.g. surface portion 17) and a node N of the wave away from the surface 17, generating a local maximum force on the bodies 9, 10 at or near the surface towards the node. Thus, as explained in detail in WO 2018/083193, incorporated herein by reference, application of the signal may serve to probe adhesion/detachment of the bodies 9 to the surface and/or objects 10 etc. on the surface in dependence of the strength of the force.

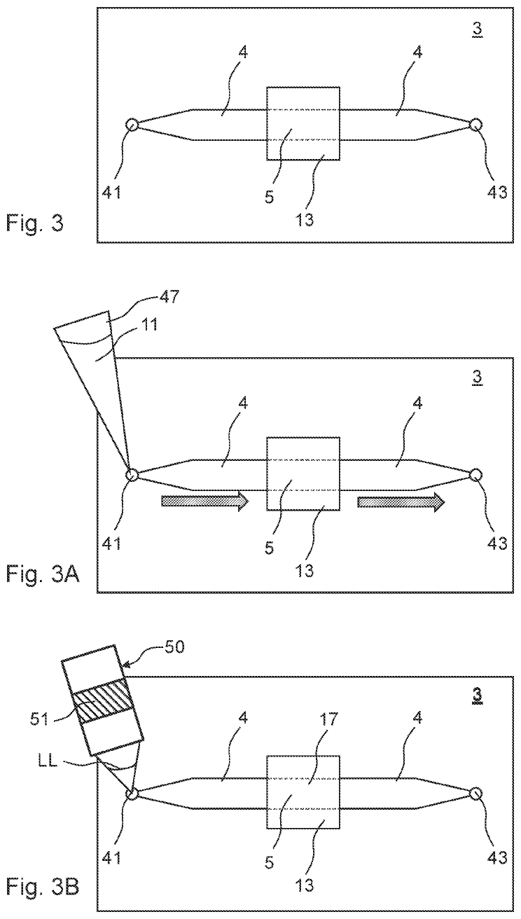

FIGS. 3-5 indicate a top view of a sample holder 3 and indicate exemplary method steps. As indicated in FIGS. 3-5, an acoustic force sample holder 3 may comprise a single channel 4. The holding space 5 may be determined by a shape variation in the channel 4, and/or by the location of the acoustic wave generator 13 and/or by the location of a window for imaging etc. overlapping part of the channel 4. In the shown embodiment, the channel 4 comprises an inlet 41 and an outlet 43 for connection to the conduits 38, 39 (cf. FIGS. 1-2).

FIG. 3A indicates filling the channel 4 and the holding space 5 with a sample liquid via the inlet 41, e.g. using the conduit 38 and/or another liquid supply 47 such as a pipette. Thus, at least part of the channel 4 is filled with the sample liquid 11. The filling may be done by allowing the sample liquid to flow into the channel 4 by gravity and/or by wetting- and/or capillary action of the channel 4 alone. Also or alternatively, at least part of the filling may be done by pressure (difference) such as by applying pressure at the inlet 41 and/or suction at the outlet 43. Preferably, the sample fills the sample channel 4 completely. When filling, bubbles may be prevented as much as possible by adjusting a flow velocity and/or by flushing accidental bubbles out of the channel 4.

During and/or after the loading, the sample liquid pressure may be ambient pressure P1.

FIG. 3B shows that, in a subsequent step, the liquid in the channel 4 is pressurised for a first time to a first elevated pressure P2, higher than the ambient pressure P1, using a compressor 50. At the outlet 43 a closure is provided to allow the pressure build-up in the channel 4 and prevent pushing sample liquid out of the channel 4. The compressor 50 may comprise a piston 51, and in the compressor and/or in an optional conduit between the compressor and the inlet 41 (not shown) an amount of gas may be provided determining a liquid level LL. The compressor may then operate on the gas to compress the gas and thereby cause increasing the sample liquid pressure to the elevated pressure P2. The sample liquid pressure is maintained at the elevated pressure P2 for a period of time, e.g. 3-5 hours, to cause dissolving of gas into the sample liquid and to destroy any bubbles. The elevated pressure P2 need not be (held) constant. Removal of the bubbles may be determined by monitoring presence and/or size of bubbles in the holding space 5. When bubbles are not or no longer detected, the sample pressure may be reduced to an operating pressure.

Establishing and maintaining the sample liquid pressure at an elevated pressure may be repeated; it is conceivable that after an initial period of maintaining the elevated pressure, the pressure is reduced but that bubbles considered to have been destroyed grow and/or reappear again to become detectable at a pressure at or near the operating pressure. Repetition of the steps of pressurising the sample liquid and maintaining the sample liquid pressure at an elevated pressure may ensure removal of all bubbles or at least all bubbles negatively affecting the sample and/or use of the sample.

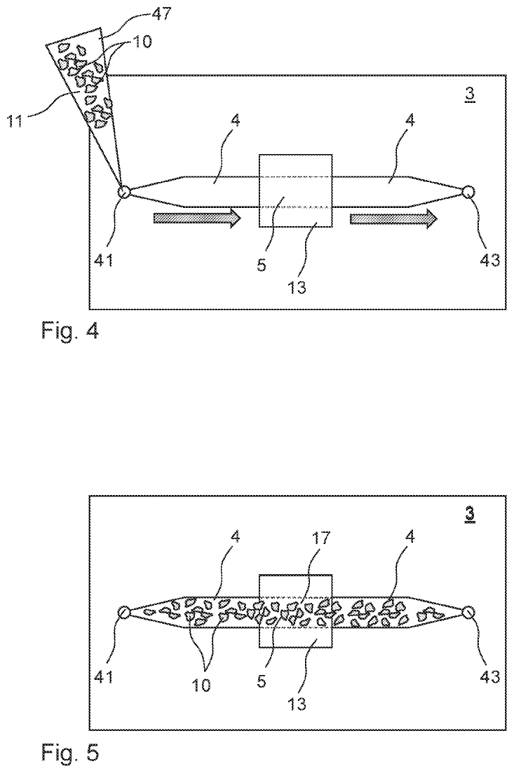

FIG. 4 and FIG. 5 indicate subsequent loading the channel 4 and the holding space 5 with a further sample liquid and optional target cells 10 via the inlet 41, e.g. using the conduit 38 and/or another liquid supply 47 such as a pipette. Thus, at least part of the channel 4 is filled with the sample liquid 11. The further sample liquid in this step may be different from the sample liquid used in the step before, but preferably being substantially the same and/or being at least miscible with the sample liquid already in the channel 4. The filling may again be done by allowing the sample liquid to flow into the channel 4 by gravity. Also or alternatively, at least part of the filling may be done by pressure (difference) such as by applying pressure at the inlet 41 and/or suction at the outlet 43. When filling, bubbles may be prevented as much as possible by adjusting a flow velocity and/or by flushing accidental bubbles out of the channel 4.

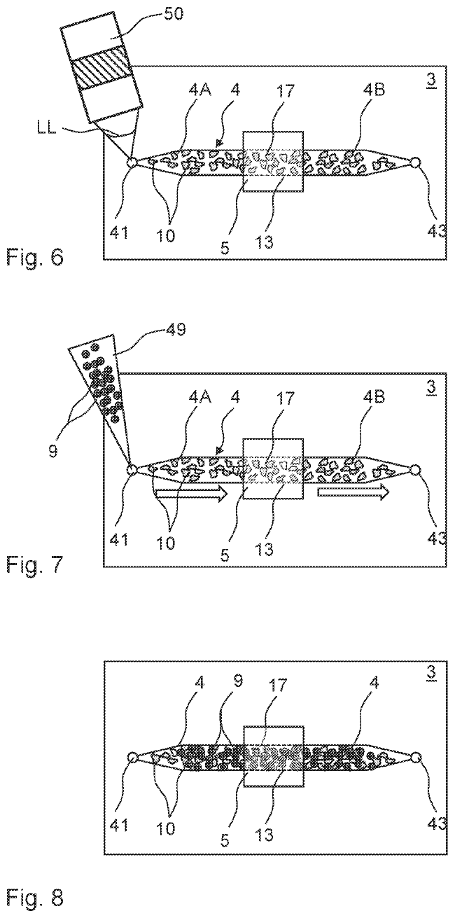

During and after the subsequent loading, the target cells 10 entrained in the sample liquid filling and flowing through the sample channel 4 are distributed over the channel 4 and the holding space 5 and left to settle there on a wall of the sample holder 3. Thus, a functionalized wall surface portion 17 is formed, see FIGS. 5 and 6 cf. FIG. 2A. Formation of the functionalized wall surface portion 17 may comprise further suitable steps such as giving the target cells 10 time to attach to the surface and grow into a monolayer. Also, a functionalized wall surface portion 17 may (be formed to) comprise different portions and/or portions with different properties.

The functionalized wall surface portion 17 may be distributed over the channel 4 beyond the holding space 5, and may extend into conduits 38 and/or 39 if used. The loaded cells 10 may be incubated and/or cultured in the channel 4. At least part of the medium may be refreshed during incubation/culturing. Flow rates over a few tens of nanoliters per minute in microfluidic channels and/or shear stress levels over 1 mPa may be avoided to reduce or prevent shear stress to the cells.

During and/or after the loading, the sample liquid pressure may be ambient pressure P1.

FIG. 6 shows that, after the loading and possibly during the incubation and/or culturing, the liquid in the channel 4 may optionally be pressurised a second time to a second elevated pressure P2-1, higher than the ambient pressure P1, using a compressor 50 (possibly the same as before), and that the sample pressure is maintained at the second elevated pressure P2-2 for a second period of time, e.g. half an hour or 1 hour. Thereafter the sample liquid pressure is reduced to a second operating pressure P3-2 which may be equal to an ambient pressure and/or the (first) operating pressure P3. The second elevated pressure P2-2 may be equal to or different from the first elevated pressure P2.

FIG. 7 indicates, as a subsequent step, loading a further sample component comprising objects 9 such as effector cells 9 or other cellular bodies in a sample liquid into the holding space 5 from a supply 49 and/or conduit 38 via the channel 4 (see arrows). During the loading, care may be taken to fill not only a lead-in section 4A of the channel 4 upstream of the holding space 5 but also a lead-out section 4B of the channel 4 downstream of the holding space 5 to ensure that the cells 9 pass through and into the holding space 5. Note that in prior art experiments, bubbles would often have been present in the channel 4 and such bubbles were found to become entrained in the fluid flow and cause damage to the functionalized layer of target cells 10. Such problems are prevented with the present concepts.

FIG. 8 indicates that the effector cells 9 may be allowed to settle and/or interact with the target cells 10. Again, the thus prepared sample may be stored and/or cultured in the channel 4 with or without (sample) liquid flow.

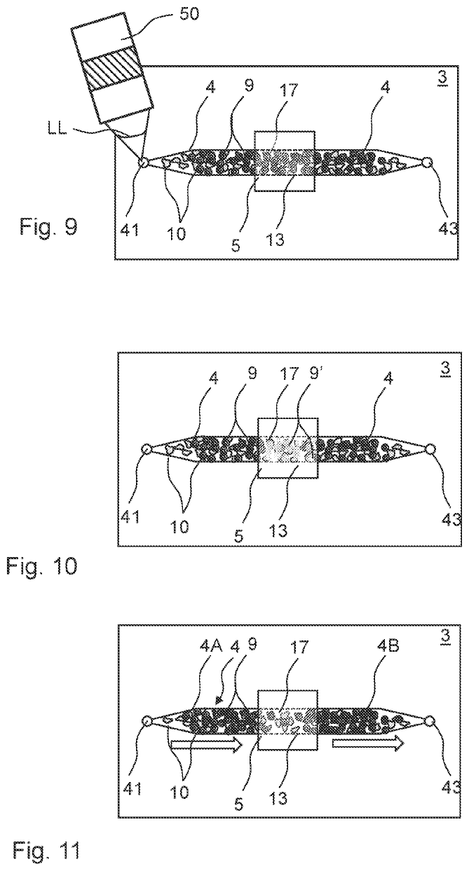

FIG. 9 indicates that, as explained before with reference to FIG. 6, the sample fluid may optionally be pressurised a third time to raise a sample liquid pressure to a third elevated pressure P2-3, and that the sample pressure is maintained at the third elevated pressure P2-3 for a third period of time, e.g. half an hour or 1 hour. Thereafter the sample liquid pressure is reduced to a third operating pressure P3-3 which may be equal to an ambient pressure and/or the (first or second) operating pressure. The third elevated pressure P2-3 may be equal to or different from the first and/or second elevated pressures P2, P2-2.

Different embodiments may comprise only a single instance of such combination of steps of pressuring the sample liquid to an elevated pressure and maintaining the elevated pressure, or two instances or rather even more such instances, in particular in case sample liquid is added and/or exchanged more times. However, multiple instances, i.e. re-application of pressure and maintaining such pressure, may not be necessary if addition of new fluids is done in a properly wetted fluid reservoir and/or if trapping of gas is avoided, this may be facilitated if sharp corners and/or rough surfaces are prevented in the reservoir where the new fluid may be introduced. Some sharp corners and rough surfaces may be difficult to avoid in the full microfluidics system, in particular for example at the interfaces between the chip and the rest of the fluidics system. An advantage of the current method is that as long as the system is fully wetted once by pressurization it may be possible to avoid or limit bubble formation at later steps without the need for further pressurization and/or other measures. This leads to more design freedom for microfluidics systems.

FIGS. 10-11 indicate exemplary use of the sample holder 3 and sample at an operating pressure P3. In particular, FIG. 10 indicates generating an acoustic wave in the holding space 5 by the acoustic wave generator 13 connected with the sample holder 3, thus exerting a force on the cellular bodies 9, 10 of the sample in the holding space 5. The acoustic force is, as a preferred option, adjusted such that part of the cells 9 are forced from the functionalized wall surface 17 towards the node in the sample liquid (indicated as cells 9′ with a lighter color), while cells 9 that are stronger bound to the functionalized wall surface 17 may remain adhered (cf. FIG. 2A). Thus, by adjustment of the force, separations may be made between different types of effector cells 9 based on adhesion characteristics; e.g. unbound—loosely bound—strongly bound.

FIG. 11, e.g., shows that the detached cells 9 may be flushed out of the holding space 5 and removed from the sample holder 3 at the outlet 43 and collected. If so desired, thereafter a further acoustic wave may be provided in the holding space 5, e.g. stronger than the first to detach stronger bound cells not detached before, and such later-detached cells 9 (i.e. having been more strongly bound) may be separately collected.

Note that the terms “inlet” and “outlet” may generally relate to the direction of a fluid flow through the respective structure, rather than specific ports of the sample holder 3, unless one or more one-way flow direction elements (valves, pumps, etc. are provided). E.g., in a variant to the process described above with respect to FIGS. 10-11, during or after application of the acoustic wave, a fluid flow direction may be reversed and the inlet 41 may serve as outlet, whereas outlet 43 may serve as inlet for liquid.

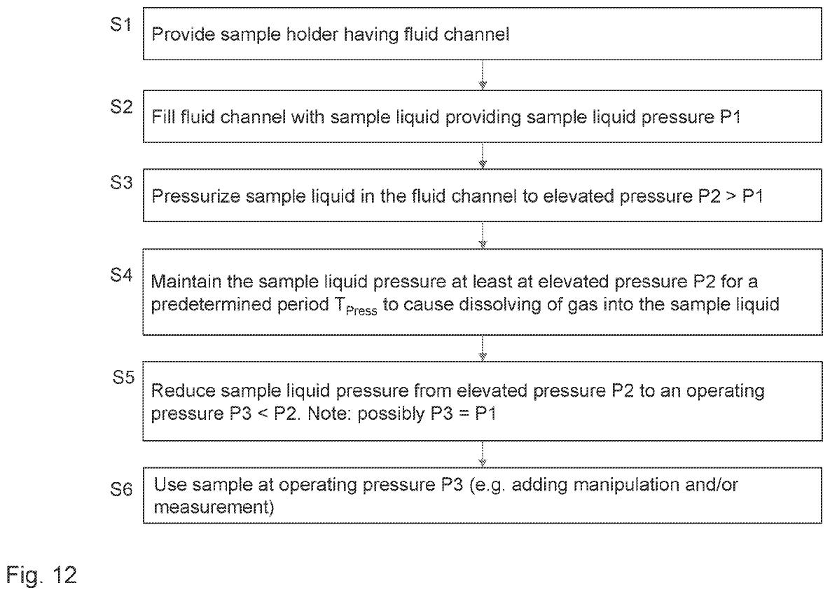

FIG. 12 is a schematic overview of method steps according to an embodiment, identified as steps S1-S6.
Step S1 indicates a step of providing a microfluidic sample holder comprising an enclosed fluid channel for holding at least part of the sample liquid.
Step S2 indicates filling at least part of the fluid channel with a sample liquid. The sample liquid thereafter is provided at a first pressure P1, e.g. ambient pressure.
Step S3 indicates pressurizing the sample liquid in the fluid channel to raise a sample liquid pressure to an elevated pressure P2 higher than the first pressure P1 higher than an ambient pressure and an operating pressure.
Step S4 indicates maintaining the sample liquid pressure at least at the elevated pressure for a predetermined period to cause dissolving of gas into the sample liquid.
Step S5 indicates reducing the sample liquid pressure from the elevated pressure to the operating pressure.
Step S6 indicates use of the sample comprising the sample liquid, having had any bubbles in it removed, at an operating pressure P3. An exemplary use comprises manipulating one or more objects in the sample liquid by an acoustic force as described herein elsewhere.

As discussed above with respect to FIGS. 3A-3B, 6 and 9, steps S2-S5 may be repeated one or more times, wherein step S2 may then comprise modifying the sample such as by changing an amount and/or composition of the sample, e.g. adding and/or exchanging sample liquid with respect to previous instances of performing step S2, and/or introducing sample particles, cf. FIGS. 4-5 and/or FIG. 7.

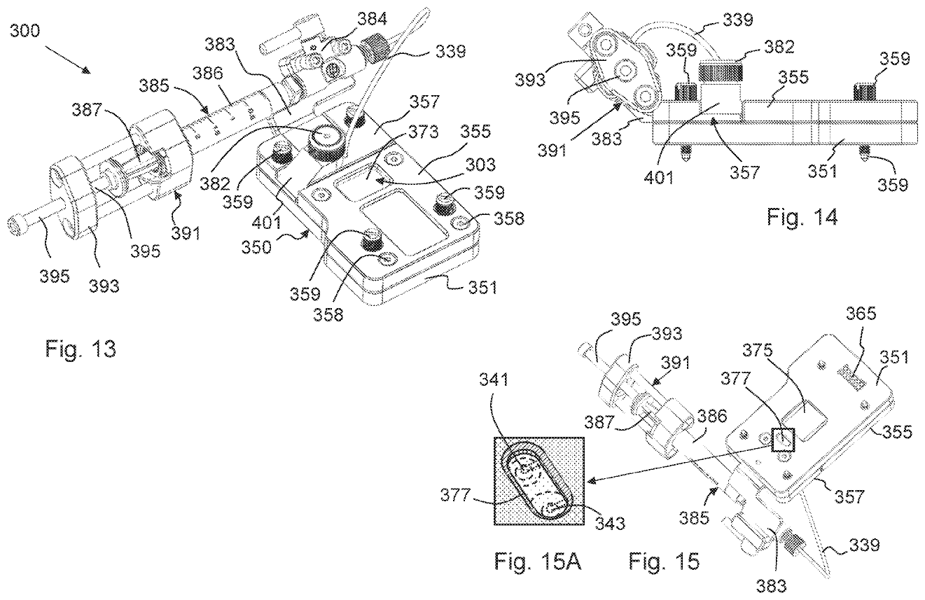

FIG. 13 shows an exemplary sample holder 300 for the system and method in perspective.

FIG. 14 is a side view of the sample holder 300.

FIG. 15 is a bottom view of the sample holder 300.

The sample holder 300 comprises a “chip” 303 in a housing 350. FIG. 15A is a detail of FIG. 15.

The shown housing 350 comprises a bottom shell 351 and an upper shell 353, which here comprises two parts, referred to as chip cover 355, and connector part 357, respectively. The housing 350 holds the chip 303.

The parts 351, 353 (=355, 357) are attached together around the chip 303, e.g. using bolts 358 as indicated, but other attachment systems could be used, e.g. clamps, and/or be permanently attached, e.g. glued or welded. It is noted that a suitable housing could comprise more or less parts and each part and/or the housing as a whole could be shaped differently than shown here. The housing 350 may be at least partly opaque. Screw bolts 359 are provided as one option for fixing the sample holder 300 to other parts of the system (not shown).

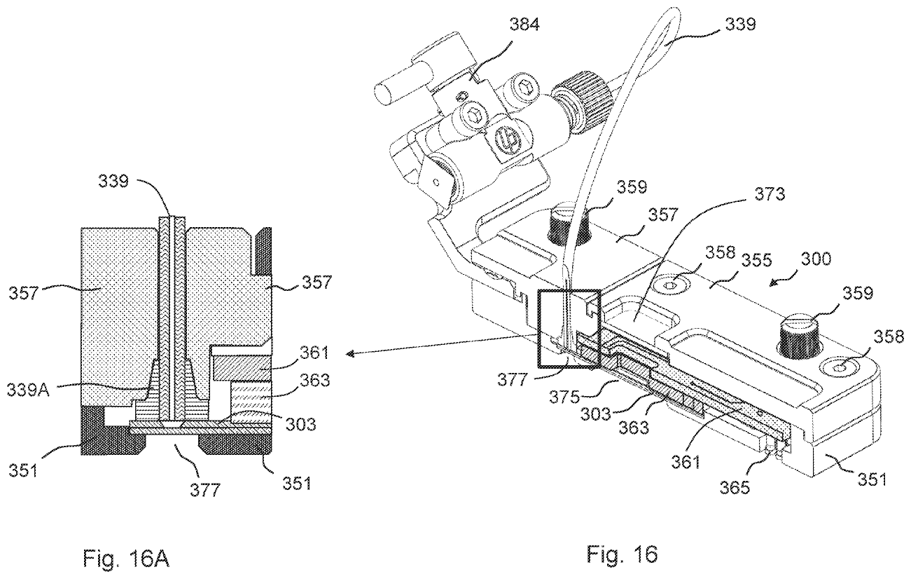

FIG. 16 is a perspective cross section view of the sample holder assembly of FIGS. 13-15, showing the sample holder or “chip” 303 in the housing 350. Also visible is that the housing 350 further accommodates an optional printed circuit board 561 (“PCB”) and an optional connector pad 363. The PCB 361 may provide any electrical connection to the chip and/or may provide other functions such as chip ID, calibration parameters and temperature control. The connector pad 363 may facilitate a thermal connection between the chip and the PCB 351. A multi-pin electrical connector 365 is provided for connecting control- and/or power signals to an optional acoustic transducer on the chip 303, (see also FIG. 15) and/or for other signals for one or more of control, power, temperature control, detection and measurement.

FIGS. 13, 15 and 16 show that the chip cover 355 comprises a first window 373 and that the bottom shell 351 of the housing 350 comprises two windows 375 and 377, respectively. The windows 373, 375 are arranged overlapping, possibly registered, and allow approaching of and/or contact and/or optical access to the chip 303 (see also below) as well as allowing that the housing 350 is opaque elsewhere. However, in the shown embodiment, the connector part 357 is transparent. The windows 373, 375, 377 are optional and if present may be formed as openings, as here, and/or one or more of them may comprise a transparent portion.

FIG. 15A is a detail of FIG. 15, as indicated, and shows that the window 377 provides optical access to and/or illumination of part of the chip 303, in particular the inlet 341 and outlet 343 of a channel 304 in the chip 303, see below.

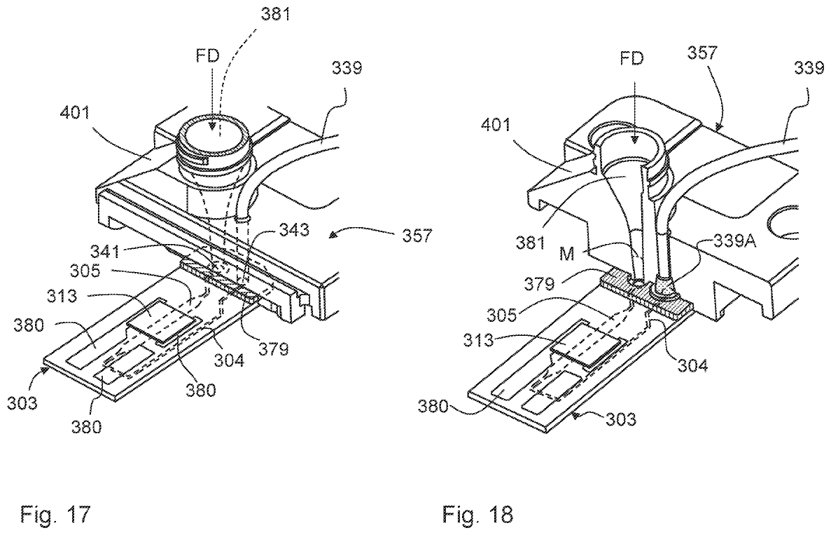

FIG. 17 shows the connector part 357 of the housing 350 and the chip 303, without the bottom shell 351 and chip cover 355. Between the connector part 357 and the chip 303 a resilient sealing gasket 379 is arranged, providing a liquid and gas tight connection between the connector part 357 and the chip 303. In dashed lines, some (not all) structures are indicated which are internal in the connector part 355, the gasket 379 and the sample holder 303, respectively.

FIG. 18 is a view like FIG. 17, but now with part of the connector part 357 cut away.

In the chip 303 a fluid channel 304 is indicated. The chip 303 may be, as shown, generally planar and the channel 304 is generally U-shaped in such plane. The channel 304 comprises a widened portion 305 which forms a holding space for a sample for experiments. The (channel 304 of) chip 303 comprises an inlet 341 and an outlet 343 for fluid sample materials. The sample holder 303 further is provided with an acoustic wave generator 313 such as a piezo element or other transducer for generating an acoustic wave in the holding space 305. FIGS. 17 and 18 also show electrical connections 380 for the acoustic wave generator 313.

The connector part 357 comprises a sample liquid reservoir 381 fluidly connected with the inlet 341 of (the channel 304 of) the chip 303. The liquid reservoir 381 is closeable gas tight with a sealed cap closure 382 (see also FIGS. 13, 14). Further, the connector part 357 provides a conduit 339 fluidly connected with the outlet 343 of (the channel 304 of) the chip 303, here via an optional ferrule 339A contained in or by the gasket 379, providing a liquid and gas tight seal (see also FIG. 16A).

Referring again to FIGS. 13-16, attached to the housing 350 is an optional mount 383 holding a valve 384. The valve 384 is connected with the chip 303 via the conduit 339.

A syringe 385, or other fluid reservoir, may be connected with the valve 384 as shown, preferably releasably connected. The syringe 385 comprises a cylinder 386 and a piston 387. In the shown embodiment, the syringe 385 is provided with an optional adjustable clamp 391. The clamp 391 and the syringe 385 are attached to each other, preferably removably attached. The shown exemplary clamp 391 comprises a mount 393 and a pusher 395 threaded into the mount 393. When the clamp 391 and the syringe 385 are operably assembled as shown, the clamp 391 can controllably depress the piston 387 into the cylinder 386 of the syringe 385 by screwing the pusher 395 into or out of the mount 393. Likewise, also or alternatively a desired relative position of the piston 387 and the cylinder 386 may be established and maintained. The assembly of the syringe 385 and the clamp 391 serves as an adjustable compressor as will be set out below.

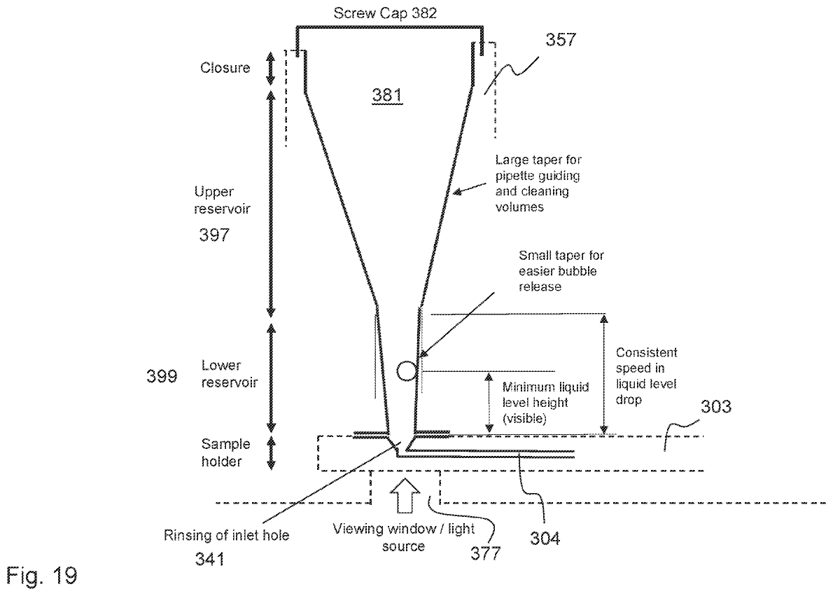

Referring back again to FIGS. 17-18 and now also referring to FIG. 19, indicating a schematic cross section profile of the reservoir 381, the reservoir 381 defines a filling direction FD, in particular wherein the reservoir 381 is substantially vertically pointing upward and widening for receiving a pipette tip (not shown) or the like for filling the reservoir with a sample liquid when the cap 382 is open or removed (cf. FIG. 17, 18). Thus, the liquid reservoir 381 comprises a tapering section having an inclined wall section facing towards the filling direction. In particular, the tapering section defines a first section 397 having a first tapering angle and a second section 399 having a second, different, tapering angle, respectively.

The first section 397 allows for easy filling as indicated above and possibly for holding relatively large amounts of liquid. This may also facilitate rinsing of the reservoir 381 (and possibly of the channel 304), e.g. for consecutive addition of different liquids, for working with valuable sample materials and/or for cleaning and reuse.

The second, relatively narrow and steep section 399 facilitates release of bubbles by the tapering shape. In the comparably narrow portion 399 small volume changes in the reservoir 381 are easier noticeable than in a comparably wider portion 397. This facilitates determining small volumes and/or filling the sample holder 303 with such small volumes (typically on the order of about 10 microliters) such as for working with valuable sample materials, e.g. (a sample comprising) patient extracted T-cells. Also, because of the steeper taper of this second section 399, for a given fluid flux there is a relatively small change in the speed of movement of the liquid level (e.g. meniscus) as the liquid level drops or rises in the second section 399, compared to in the first section 397 that is wider and has less steep walls. E.g. during emptying of the reservoir, due to the steep taper of the second section 399 there is only a small acceleration of the liquid level drop as the liquid level approaches the minimum visible liquid level height. This facilitates better control over the liquid level by the user. Bubbles introduced by pipetting or nucleation may be removed by releasing from the wall. Bubbles may release better due to the inclined wall upward facing the liquid level in the filling direction provided by the slight taper, compared to a vertical wall such as in a non-tapered straight reservoir. This may be because chances are reduced that a bubble contacts the wall just released and/or, as the bubble in contact with the reservoir wall rises up there is a smaller chance to contact the opposite wall. This facilitates bubble release. The taper, of the first and/or of the second section may also facilitate guiding a filling device such as a pipette tip to the inlet 341 of the sample holder 303 for delivery of sample material close to the sample holder. This may reduce accumulation or remaining of sample objects in the reservoir 381 which might interfere with measurements and/or manipulation in the channel 304 later on, e.g. when further sample liquids are introduced.

The reservoir 381 may be provided with one or more level marks M for reference (FIG. 18).

The connector part 357 provides a window 401 for optical detection, in particular visual detection, of a liquid level and/or a level mark in the reservoir 381. The window 401 also allows the user or the system to detect potential bubble issues, in particular by allowing inspection close to the bottom of the reservoir and/or the inlet hole 341 of the chip. For that, at least part of the connector part 357 is transparent, possibly all of the connector part 357, as in the shown embodiment. Preferably most of the reservoir 381 if not all of it is visible through the window 401. The window 401 may be plane or be curved or otherwise formed to provide lens action for magnification and/or otherwise facilitating detecting a liquid level in the reservoir. The orientation of the window 401 and/or further more or less conspicuous optical indicators may urge a user to adopt a predetermined viewing angle and/or direction, thus increasing consistency between detections and reliability of the procedure.

Due to the translucency and/or transparency of the connector part 357 level indication is facilitated, which may be further assisted by the window 377 enabling access of light “from below”.

An exemplary method of filling the channel 304 of the sample chip 303 in the sample holder 300 comprises the following steps:
A—Filling the syringe 385 with a known volume of a gas, e.g. 0.5 ml of air. The syringe 385 may be detached from the valve 384 for this. The syringe 385 should then be connected with the valve. Note that a three-way valve (or more than three ways) could be used for filling the syringe with another gas and/or for filling the syringe 385 without removal from the valve 384.
B—Filling the liquid reservoir 381 with a known quantity of a sample liquid. E.g., 0.3 ml of aqueous solution Phosphate Buffered Saline (PBS). The known quantity may be determined by a level mark in the reservoir 381 and/or by using a calibrated pipette. Preferably, bubble formation during filling is prevented or minimised.
C—Filling the sample channel 304 with the sample liquid. In the present method this is done by suction of the liquid through the channel using the syringe 385 by retracting the piston (and setting the valve 384 open between the syringe 385 and the sample holder 303). The desired amount of retraction may be determined from indications on the syringe but it is preferred that (receding of) a liquid level in the reservoir 381 to a predetermined height (possibly indicated with a level mark on or in (the reservoir 381 of) the system 300 is used as a gauge. The resultant syringe configuration is maintained thereafter until in step F below.
D—Re-filling the liquid reservoir 381 with a further known quantity of a sample liquid. E.g., again 0.3 ml of aqueous solution PBS. The known quantity may be determined by a level mark in the reservoir 381. Preferably, bubble formation is prevented or minimised. The valve 384 may remain open or be closed between the syringe 385 and the chip 303.
E—Close the reservoir 381 liquid- and gas tight with the sealing cap 382. The valve 384 may be open or closed.
F—Pressurize the sample liquid in the fluid channel 304 using the syringe 385 provided with the clamp 391. This may comprise one or more of the following sub-steps:
F1—Ensure that the conduit is liquid-filled and that a liquid level in the syringe 385 perpendicular to the conduit entrance, e.g. by orienting the syringe vertical with the piston up and the syringe-outlet down, so that no bubbles are accidentally introduced into a conduit and/or the channel.
F2—If not already done so previously, attach the clamp 391 to the syringe 385.
F3—While maintaining the orientation discussed in substep F1, with the valve 384 open compress the gas in the syringe 385 by depressing the piston 387 for a predetermined amount and fixing the piston 387 at the desired position to pressurise and maintain the gas at the thus established desired elevated pressure. Note that by closing off of the reservoir 381 in substep E the pressure may be built up since the opposite ends of the sample holder are sealed. In case of a sample holder comprising plural interconnected channels inside the chip, all in- and outlets should be sealed liquid- and gas tight for the pressurization.

Note that pressurisation may be applied from either an inlet-side or an outlet-side of the channel. E.g. pressurisation could also be provided from the side of or on the reservoir. In such case, the same or a further assembly comprising a syringe and a clamp could be connected with (a modified version of) the cap 382 (not shown), possibly via a suitable port on a multi-port valve replacing the shown valve 384.
G—Maintaining the sample liquid pressure at least at the elevated pressure for a predetermined period to cause dissolving of gas into the sample liquid. Particularly if the liquid level of the thus-pressurised system is on the sample holder side of the valve 384 instead of on the syringe side, the valve may also be closed to maintain the elevated pressure without having to rely on the syringe and clamp.
H—Reducing the sample liquid pressure from the elevated pressure to the operating pressure by relaxing the clamp and retracting the piston from the syringe gently to the desired position associated with the operating pressure. This may mean retracting the piston to the position at the end of step C above.

The disclosure is not restricted to the above described embodiments which can be varied in a number of ways within the scope of the claims.

For instance another compressor than a syringe with or without a clamp may be used. The compressor may be computer-controlled. Also, the sample holder may be designed and/or used for other types of microfluidic experiments and/or purposes than acoustic force measurements and/or optical tweezers experiments.

Elements and aspects discussed for or in relation with a particular embodiment may be suitably combined with elements and aspects of other embodiments, unless explicitly stated otherwise.

1. A method of reducing or preventing bubbles in a microfluidic sample liquid, comprising:
providing a microfluidic sample holder comprising an enclosed fluid channel for holding at least part of the sample liquid,
filling at least part of the fluid channel with the sample liquid,
closing the fluid channel and pressurizing the sample liquid in the closed fluid channel to raise a sample liquid pressure to an elevated pressure higher than an ambient pressure and an operating pressure,
maintaining the sample liquid pressure at least at the elevated pressure for a predetermined period to cause dissolving of gas into the sample liquid, and
reducing the sample liquid pressure from the elevated pressure to the operating pressure.

2. The method according to claim 1, wherein the operating pressure is equal to the ambient pressure.

3. The method according to claim 1, further comprising providing an amount of gas in a limited volume in contact with the sample liquid providing a liquid level;
wherein at least one of pressurizing the sample liquid in the fluid channel and maintaining the sample liquid pressure at or above the elevated pressure for a predetermined period comprises compressing the gas in the limited volume.

4. The method according to claim 1, wherein the elevated pressure is in a range of about 1-20 Bar (100-2000 kPa) over ambient pressure.

5. The method according to claim 1, further comprising adding an additional amount of sample liquid to the sample liquid.

6. The method according to claim 1, wherein the sample liquid comprises sample particles.

7. The method according to claim 1, comprising providing at least part of the fluid channel with a functionalised wall surface portion.

8. The method according to claim 1, further comprising providing a signal generator for generating an acoustic wave in the sample holder; and
providing, using the signal generator, a driving signal to the sample holder generating an acoustic wave in the sample holder.

9. The method according to claim 1, further comprising providing an optical trapping beam for trapping and/or manipulating at least part of a sample in at least part of the channel.

10. A system for filling a microfluidic sample holder, wherein the sample holder comprises
an enclosed microfluidic channel provided with a liquid inlet and a liquid outlet and a closure for closing the channel gas- and liquid-tight;
a filling system for filling at least part of the microfluidic channel with a sample liquid; and
wherein the filling system comprises a controller and a compressor and is configured for
controllably pressurizing a sample liquid in the channel when closed, to raise a sample liquid pressure to an elevated pressure higher than an ambient pressure and an operating pressure,
maintaining the sample liquid at the sample liquid pressure at least at the elevated pressure for a predetermined period to cause dissolving of gas into the sample liquid for removing from and/or preventing gas bubbles in the sample liquid, and
controllably reducing the sample liquid pressure from the elevated pressure to the operating pressure.

11. The system according to claim 10, comprising a sealed or sealable gas reservoir for providing a defined amount of gas in a limited volume in contact with the sample liquid providing a liquid level.

12. The system according to claim 11, wherein the compressor is configured to compress a gas in the gas reservoir to pressurize the liquid.

13. The system according to claim 10, comprising a liquid reservoir in fluid connection with the channel,
wherein the liquid reservoir defines a filling direction and at least part of the liquid reservoir comprises a section having an inclined wall section facing towards the filling direction.

14. The system according to claim 10, wherein the sample holder comprises a translucent or transparent portion for optical detection of a liquid level, and
wherein the translucent or transparent portion may be provided with a level mark and/or allow optical access to a level mark, and/or at least part of the translucent or transparent portion is formed as a light guide.

15. The system according to claim 10, wherein the sample holder comprises an at least partly opaque portion at or near the liquid inlet and/or the liquid outlet,
wherein the opaque portion provides a translucent or transparent portion and/or an aperture, for lighting at least one of a liquid inlet of the channel and a liquid outlet of the channel;
wherein in particular in a system according to claim 12 the translucent or transparent portion and/or an aperture is separate from the translucent or transparent portion for optical detection.

16. The system according to claim 13, comprising a sealed or sealable gas reservoir for providing a defined amount of gas in a limited volume in contact with the sample liquid providing a liquid level; and a closure for closing the gas reservoir and/or the liquid reservoir.

17. The system according to claim 10, wherein the sample holder comprises an acoustic wave generator for generating an acoustic wave in at least part of the channel, and/or the system comprises at least one optical tweezer for trapping and/or manipulating at least part of a sample in at least part of the channel.

18. The system according to claim 10, wherein the elevated pressure is in a range of about 1-20 Bar (100-2000 kPa) over ambient press.

19. The method according to claim 8, wherein generating the acoustic wave in the sample holder provides an acoustic force in at least part of the channel for manipulating one or more objects in the sample liquid.

20. The system according to claim 13, wherein the inclined wall section defines a first section having a first inclination and a second section having a second, different inclination.
